(19) United States
(12) Patent Application Publication
(10) Pub. No.: US 2009/0093791 A1
(43) Pub. Date: Apr. 9, 2009

(54) DEVICES AND METHODS FOR TREATING CHRONIC TOTAL OCCLUSION

(76) Inventor: Richard R. Heuser, Phoenix, AZ (US)

Correspondence Address: KOLISCH HARTWELL, P.C., 200 PACIFIC BUILDING, 520 SW YAMHILL STREET, PORTLAND, OR, 97204, US

(21) Appl. No.: 12/336,401

(22) Filed: Dec. 16, 2008

Related U.S. Application Data
(63) Continuation-in-part of application No. 11/433,198, filed on May 11, 2006.
Application No. 11/433,198 is a continuation-in-part of application No. 10/272,317, filed on Oct. 15, 2002, now Pat. No. 7,179,250.
Application No. 10/272,317 is a continuation of application No. 09/705,963, filed on Nov. 3, 2000, now Pat. No. 6,464,681.
Application No. 09/705,963 is a continuation of application No. 09/397,806, filed on Sep. 17, 1999, now Pat. No. 6,159,197.

(51) Int. Cl. A61M 25/10; A61B 18/18

(52) U.S. Cl. 604/509; 606/41; 604/103.01

(57) ABSTRACT

Catheterization systems and methods for treatment of a condition within a blood vessel are provided that include the use of a catheter, a balloon immediately adjacent to the distal end of the catheter, an inflation device for expanding the balloon, and an occlusion-penetrating device for gaining access through an occlusion. The occlusion-penetrating device may include an indeflator configured to injected fluid at a high pressure, an RF wire, a hollow needle wire, a dissection tool, a laser wire, or even a very small balloon to exploit existing microchannels in the occlusion.

### RELATED APPLICATIONS

This application is related as a continuation-in-part and claims priority to co-pending application Ser. No. 11/433,198, filed May 11, 2006, which is a continuation-in-part of application Ser. No. 10/272,317, filed Oct. 15, 2002, which issued on Feb. 20, 2007 as U.S. Pat. No. 7,179,250, and which was a continuation of application Ser. No. 09/705,963, filed Nov. 3, 2000, which issued on Oct. 15, 2002 as U.S. Pat. No. 6,464,681 and which was a continuation of U.S. patent application Ser. No. 09/397,806, filed Sep. 17, 1999, which issued on Dec. 12, 2000 as U.S. Pat. No. 6,159,197, all of which are incorporated herein by reference.

### BACKGROUND AND SUMMARY OF THE INVENTION

This invention relates to a device and methods for treating a condition in a blood vessel, typically an artery, where plaque and/or other buildup or constriction has caused a complete or near-complete blocking or occlusion of the blood vessel. Typically the device is for treatment of such a condition of vascular occlusion that has existed for a period of at least a month and in some cases several months or years, although it may also be used in conditions of a shorter duration. The artery may be located anywhere in the body, typically in the legs, neck, brain or heart.

Treatment of heart disease has traditionally been a highly traumatic endeavor. For many years surgeons would be required to conduct major surgery to correct even relatively minor conditions. Such “open-heart” operations are highly traumatic for the patient and may therefore not be an option for those whose bodies cannot withstand such trauma. Open-heart operations are also expensive and may be risky. There is also a possibility of the patient contracting an infection during his or her extended stay in a medical care facility. For these reasons, some conditions may not merit treatment if open-heart surgery is required for their treatment.

The use of low-trauma surgery devices and techniques has increased the treatment and success rates for many conditions that are either too risky or too expensive to perform during open-heart surgery. The catheter is one such low-trauma device that has been especially successful in the treatment of cardiovascular and other conditions. A typical catheter is a flexible, hollow small-diameter tube that is threaded through a body system (such as the cardiovascular system) until it reaches a location that requires treatment. An advantage of a catheter is that only a small incision need be made to insert the catheter into the body. This significantly reduces the trauma experienced by the patient and dramatically reduces recovery time. Furthermore, depending on the procedure, only local anesthesia may be needed. This reduces the risk and cost of the procedure. Catheters have been successfully used in angioplasty procedures and in the delivery of stents and other medical devices into selected areas of the body.

One procedure that has met with limited success using low-trauma surgical techniques is the killing off or elimination of tissues such as the septum of the heart. If a tissue-killing substance such as alcohol is inserted into an artery leading to the septum, there is a risk that some of the alcohol may travel instead through arteries leading to other portions of the heart. This would damage other portions of the heart, and a heart attack may result. Known infusion techniques have not been able to reliably deliver alcohol to a desired tissue while preventing the alcohol from damaging other tissue.

Another aspect of the invention provides a method of introducing a tissue-killing substance into a bodily fluid vessel. According to the method, a catheter is provided that has a blocking mechanism configured to selectively block and unblock the vessel. The catheter also has a delivery system that is configured to introduce the tissue-killing substance into the vessel. The vessel is substantially blocked upstream of a selected tissue using the blocking mechanism. The tissue-killing substance is introduced into the vessel through the delivery system, and the vessel is unblocked when the tissue-killing substance has substantially traveled toward the selected tissue.

The blocking mechanism may also be used to apply treatment to a vascular occlusion. The treatment may include infusion of liquid and/or the application of energy including radio-frequency, laser, or mechanical force. Vascular occlusions are more difficult to remove where the blockage includes a mineral component, typically a calcification. Such occlusions are difficult to reopen and, even if reopened, tend toward restenosis, i.e., a repeat of the occlusion. Treatment of the plaque and calcification with an appropriate substance will allow the reopening and reduce the chances of restenosis.

U.S. Pat. No. 6,290,689, which is incorporated herein by reference, discloses a catheter device for the treatment of calcified vascular occlusions.

### BRIEF DESCRIPTION OF THE DRAWINGS

FIG. 1 is a side elevational, partial cutaway view of a catheter with a balloon according to an embodiment of the invention.

FIG. 2 is a side elevational, cutaway view of a body fluid vessel with the catheter of FIG. 1 inserted therein on a guidewire.

FIG. 3 is a side elevational view of the catheter of FIG. 1 inserted in a blood vessel, the balloon inflated, and infusing a liquid into the vessel.

FIG. 4 is a side elevational, cutaway view of the catheter shown in FIG. 1 in a blood vessel with the balloon inflated adjacent an occluded portion of the vessel.

FIG. 5 is a side elevational, partially cutaway view of the catheter of FIG. 1 with a needle-tipped, hollow wire within an internal lumen of the catheter.

FIG. 6 is a side elevational, partially cutaway view of the catheter with the needle-tipped, hollow wire of FIG. 5 extending out of an aperture at the distal end of the catheter.

FIG. 7 is a side elevational, partially cutaway view of the catheter of FIG. 1 with a radio-frequency wire within an internal lumen of the catheter.

FIG. 8 is a side elevational, partially cutaway view of the catheter of FIG. 1 with a hollow wire within an internal lumen of the catheter, and a needle wire within the hollow wire.

FIG. 9 is a pictorial view of the proximal and distal ends of the needle-tipped, hollow wire of FIG. 5 showing the needle-tip at the distal end and a syringe coupled to the proximal end.

FIG. 10 is a side elevational, cutaway view of the radio-frequency wire treating an occluded portion of a blood vessel.

FIG. 11 is a side elevational, cutaway view of the needle-tipped, hollow wire treating an occluded portion of a blood vessel.

FIG. 12 is side elevational view of a catheter in accordance with an embodiment of the invention showing an ovally-shaped balloon.

FIG. 13 is a side elevational, cutaway view of the catheter with a wire having a dissection tool at its distal tip.

FIG. 14 is a side elevational, cutaway view of the dissection tool treating an occluded portion of the blood vessel.

FIG. 15 is a cross-sectional view of an occlusion that includes a plurality of microchannels.

FIGS. 16A-C depict a method of using pressurized fluid to expand one or more microchannels of an occlusion.

FIGS. 17A-B depict a method similar to that shown in FIGS. 2A-C, except that a needle is inserted through the catheter and solution is injected distally of the proximal end of the occlusion, expanding a microchannel.

FIGS. 18A-B depict another method of expanding existing microchannels to gain access through an occlusion, except that instead of fluid being injected, a wire with a very small balloon is inserted so that the very small balloon is expandable within a microchannel, to gain access through the occlusion.

FIG. 19 depicts an alternative method of stabilizing a support catheter within a blood vessel using a side branch of the blood vessel.

FIG. 20 depicts an example elastomeric membrane that may be used with the methods and systems described herein.

### DETAILED DESCRIPTION

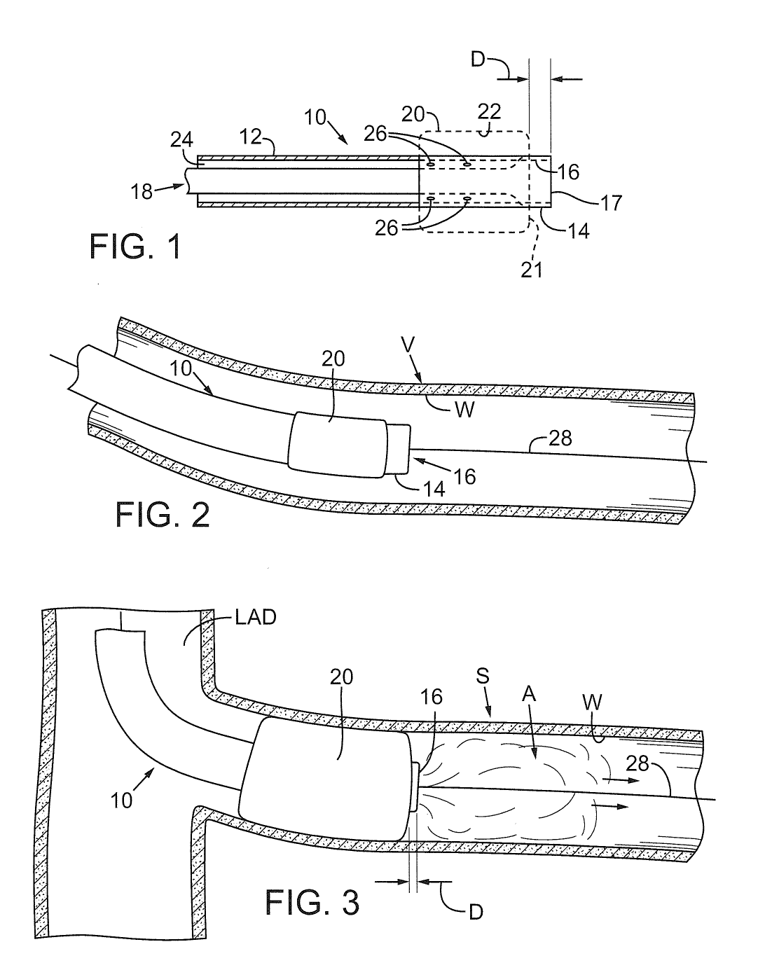

FIG. 1 depicts a first catheter 10 that may be used with the processes and procedures disclosed herein. First catheter 10 includes a flexible, generally cylindrical length of hollow tubing 12. The tubing preferably has an outside diameter of about 1-4 mm. A distal end 14 of the first catheter has an opening or aperture 16, which is defined by an annular rim or edge 17. A first passage, shown as a first lumen 18, runs the length of catheter 10 and communicates with aperture 16. First lumen 18 preferably has an inner diameter of about 0.014-0.038 inches. The first lumen permits fluids or colloids to be selectively introduced into a vessel, as will be described below.

A first flexible membrane, shown as a first balloon 20, is secured to tubing 12 adjacent distal end 14. First balloon 20 includes a distal end 21 that is preferably positioned at a distance D from rim 17 such that distal end 21 of balloon 20 is immediately adjacent aperture 16. As can be seen in FIG. 1, distance D is typically about one-half of the diameter of tubing 12, or about 0.5 mm to 2.0 mm. Alternatively, balloon 20 may be positioned with its distal edge closer to or farther from rim 17, depending on the desired application for the catheter.

First balloon 20 has an interior 22 that varies in volume when expanded and contracted. A second passage, shown as a second lumen 24, runs the length of first catheter 10 and communicates with interior 22 of the first balloon through intermediate apertures 26 that pass through tubing 12. A controlling fluid (not shown) flows within second lumen 24 and is controlled by an operator to expand/inflate and contract/deflate the first balloon. The first balloon functions as a flow-blocking mechanism to block the flow of blood or other fluid through a vessel while a surgical technique or process is being completed. As such, first balloon 20 is very compliant and inflates with a very slight change in pressure within second lumen 24. First balloon 20 preferably has an outer diameter of about 2-8 mm when fully inflated.

FIG. 2 shows catheter 10 inserted in a blood vessel V, typically an artery or vein, that is defined by a vessel wall W. Catheter 10 is threaded on a guidewire 28 that typically is inserted first into the blood vessel and maneuvered until the guidewire reaches a treatment site. Then the physician advances catheter 10 along guidewire 28 to place distal end 14 and aperture 16 of catheter 10 at the treatment site.

FIG. 3 shows catheter 10 with aperture 16 positioned at a treatment site. In this example, catheter 10 is used to kill or eliminate a desired tissue. For instance, in a case of idiopathic hypertrophic subaortic stenosis or if the septum of the heart is diseased, it may be necessary or desirable to kill the tissues comprising the septum of the heart. This may be accomplished by inserting a tissue-killing substance, such as alcohol, into the septum. First catheter 10 provides a way for such an alcohol infusion process to be performed without endangering the life of the patient. As shown in FIG. 3, the distance D between the distal end of balloon 20 and catheter distal aperture 16 may be selected for the particular application and may be smaller than that shown in FIGS. 1 and 2.

To perform this procedure, guide wire 28 is placed into the left anterior descending (LAD) coronary artery of the heart and into a septal branch S of the LAD artery (FIG. 9). First catheter 10 is guided along guide wire 28 until first balloon 20, in a contracted state, has entered septal branch S. The operator inflates first balloon 20 as previously described. An amount of alcohol A is released or delivered through first lumen 18 into septal branch S and is permitted to flow toward the septum (not shown), where the alcohol kills the tissue of the septum.

First balloon 20 serves as a blocking mechanism to prevent the flow of alcohol A out of the septal branch and into the LAD artery, where the alcohol would otherwise flow and destroy other tissues in the heart. By pressing against the interior wall W of septal branch S, first balloon 20 holds first catheter 10 in place while the alcohol is infused into the septal branch. Aperture 16 is located immediately adjacent first balloon 20, which enables an accurate delivery of alcohol relative to the first balloon. The operator completes the alcohol infusion process by deflating first balloon 20 and removing first catheter 10 and guide wire 28 from septal branch S and LAD artery.

It may sometimes be necessary to provide an electrical impulse to the heart after the alcohol infusion process is complete. This “pacing” of the heart may be accomplished by transmitting the electrical impulse through guide wire 28 prior to removing the guide wire from the septal branch or the LAD artery.

Another condition that catheter 10 may be used to treat are occlusions of blood vessels, including a chronic total occlusion which is a 100% blockages of a blood vessel that has been in existence for a significant time, typically clinically defined as 30 days or more. Catheter 10 may also be used in treating occlusions that have been in existence for a shorter period of time. Typically an occlusion becomes increasingly calcified the longer it remains in existence.

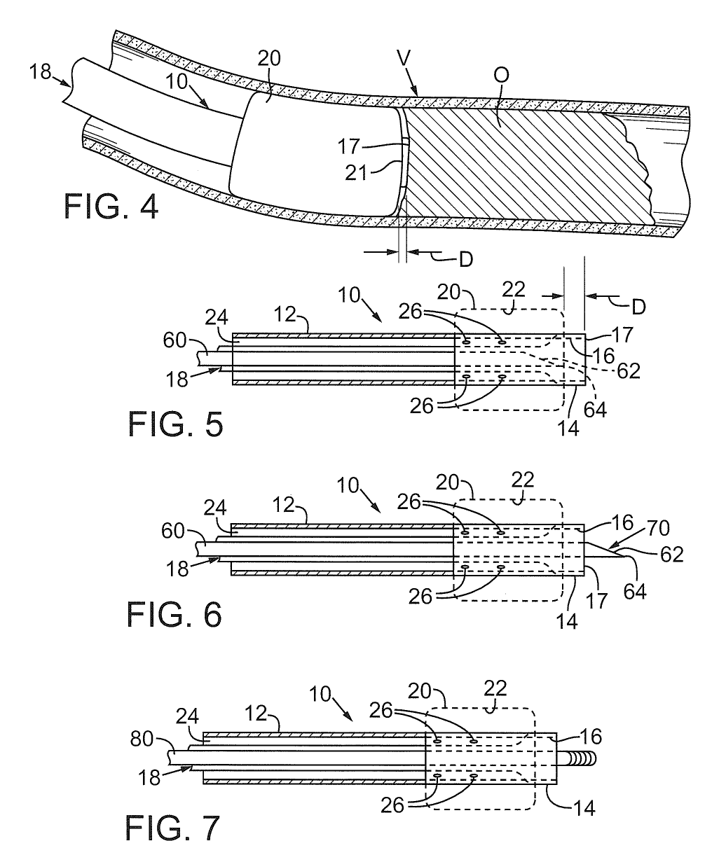

Catheter 10 is shown in FIG. 4 positioned at a treatment site for a total occlusion O. Preferably, treating such total occlusion with a liquid will involve confining the liquid to the tissue, plaque, and calcification of the total occlusion because, like the alcohol treatment described above, the liquid may be harmful to other tissue. Catheter 10 is preferably positioned with distal edge 17 butted up against occlusion O and balloon 20 is inflated. Balloon 20 holds the catheter in place and prevents the catheter from being inadvertently moved during a process.

Balloon 20 substantially seals off the wall W of vessel V proximal to distal end 14 of catheter 10. Balloon 20 also confines any liquid pumped through lumen 18 and out of aperture 16 to the tissue, plaque, and calcification of the occlusion. Some liquid may enter the area of vessel V between distal end 21 of balloon 20 and occlusion O. However, this area is limited by the separation D between distal end 21 of balloon 20 and distal edge 17 of catheter 10.

Two methods for treating a total occlusion are: (1) promoting the growth of collateral blood vessels and (2) dissolving the plaque and calcification to reopen the blood vessel. Either of these approaches may be carried out by the injection of a liquid through lumen 18 and out of aperture 16 of catheter 10 to infuse the occlusion. Promotion of collaterals may be carried out by infusion with a vascular endothelial growth factor (VEGF), a fibroblast growth factor (FGF), or such other substances that tend to promote angiogenesis.

Dissolving the plaque and calcification may be carried out by infusion of a plasminogen activator, such as urokinase or thrombolytic plasminogen activator (tPA), or other thrombolytics or other solutions that will help in breaking up the occlusion. The liquid may be injected into the total occlusion and held there by maintaining inflation of the balloon to seal off the area outside the treatment site and protect other tissue from the liquid. The time period for holding the liquid in place may be selected for the expected resistance of the plaque and calcification to the desired dissolving. For example, the liquid may be flushed in and held in place, for a short period, such as 15-20 minutes, for an intermediate period of 2-3 hours, or a long period of 12-48 hours. Typically, after the liquid treatment is completed, the occlusion, or what is left of it, will be further treated by advancement of a wire through the occluded area. Alternatively, the liquid treatment and wire advancement may be performed together, i.e., advancing a wire while the liquid is still in place, or iteratively, i.e. advancing the wire partially through the occlusion, injecting more liquid, advancing the wire further, etc.

FIGS. 5, 6, 9, and 11 show a needle-tipped, hollow wire 60 for use with catheter 10. Wire 60 is typically inserted in lumen 18 of catheter 10. Wire 60 may be positioned, as shown in FIG. 5, so that a distal tip 62 of wire 60 does not extend beyond distal edge 17 of catheter 10. This is the preferred position for advancing catheter 10 in a blood vessel so that tip 62 does not cause trauma to the vessel. Distal tip 62 of wire 60 culminates in a sharp point 64. FIG. 6 shows wire 60 with distal tip 62 extending beyond distal end 14 of catheter 10, which is the typical position at the treatment site.

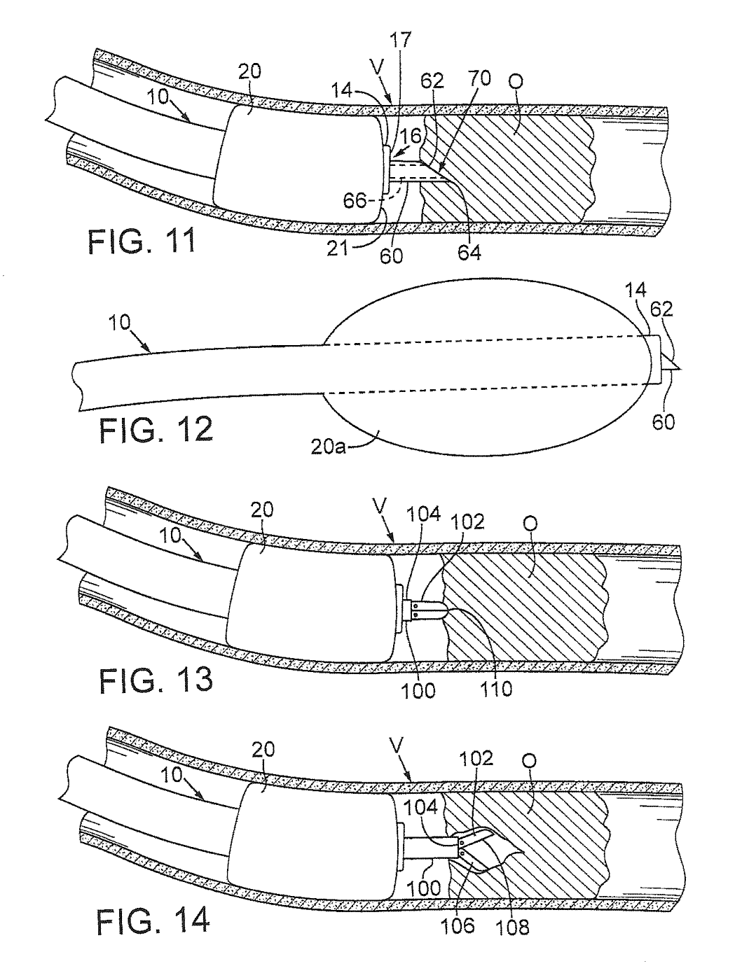

As best seen in FIGS. 9 and 11, wire 60 includes a lumen 66 extending from a proximal end 68 to a distal opening 70 at tip 62. Proximal end 68 of wire 60 may be coupled to an injection device, such as syringe 72 by a vacuum seal 74. A plunger 76 in syringe 72 may be depressed to inject a liquid through wire 60 and out distal tip 62, or plunger 76 may be withdrawn to create a vacuum to draw liquid into wire 60 at the distal tip.

FIG. 11 shows distal tip 62 of wire 60 extended beyond distal end 14 of catheter 10 and sharp point 64 inserted into occlusion O. Balloon 20 is inflated to seal off the treatment site. Liquid injected into occlusion O through wire 60 exits the wire at distal opening 70 and thus enters occlusion O at a depth within the plaque and calcification that is determined by the depth of insertion of distal tip 62 and the pressure with which the liquid is injected. The liquid is maintained in the treatment site by balloon 20 as described above. The position of distal tip 62 of wire 60 relative to distal edge 17 of catheter 10 may be selected and adjusted as desired by the physician, resulting in more or less area in the vessel between distal edge 21 of balloon 20 and occlusion O. In any case, the position of distal edge 21 of balloon 20 immediately adjacent aperture 16 and distal edge 17 of catheter 10 allows the physician to reduce the area as much as is desired.

As the occlusion is dissolved by infused liquid, wire 60 may be advanced through occlusion O. When tip 62 of wire 60 passes all the way through occlusion O, a stent and/or balloon catheter may be advanced through occlusion O and expanded to reopen the blood vessel.

FIGS. 7 and 10 show a wire 80 inserted through catheter 10. The position of wire 80 relative to catheter 10 is controlled by the physician and wire 80 may be withdrawn into lumen 18 of catheter 10 or extend beyond distal end 14 as shown in FIGS. 7 and 10. Wire 80 may be provided with a hot tip, or radio-frequency (RF) tip 82 which may be of the type described in U.S. Pat. No. 6,190,379, which is incorporated herein by reference. Wire 80 may also be provided with a lumen 84 (FIG. 10), as for wire 60, for the injection of liquids through wire 80 for infusion at a treatment site. In some embodiments, wire 80 is simply a standard wire.

Wire 80 is shown in FIG. 10 at a treatment site for an occlusion O. Balloon 20 is inflated to prevent infused liquid or debris from use of the RF tip from leaving the treatment site. RF tip 82 is shown extending completely beyond distal end 14 of catheter 10, but the tip may be moved to any position relative to catheter 10 for a desired treatment. For example, wire 80 could be withdrawn so that less of tip 82 extends beyond distal end 14 to further confine infused liquid and/or debris.

As the occlusion is dissolved by infused liquid and/or ablated by the RF tip, wire 80 may be advanced through occlusion O. When tip 82 of wire 80 passes all the way through occlusion O, a stent and/or balloon catheter may be advanced through occlusion O and expanded to reopen the blood vessel.

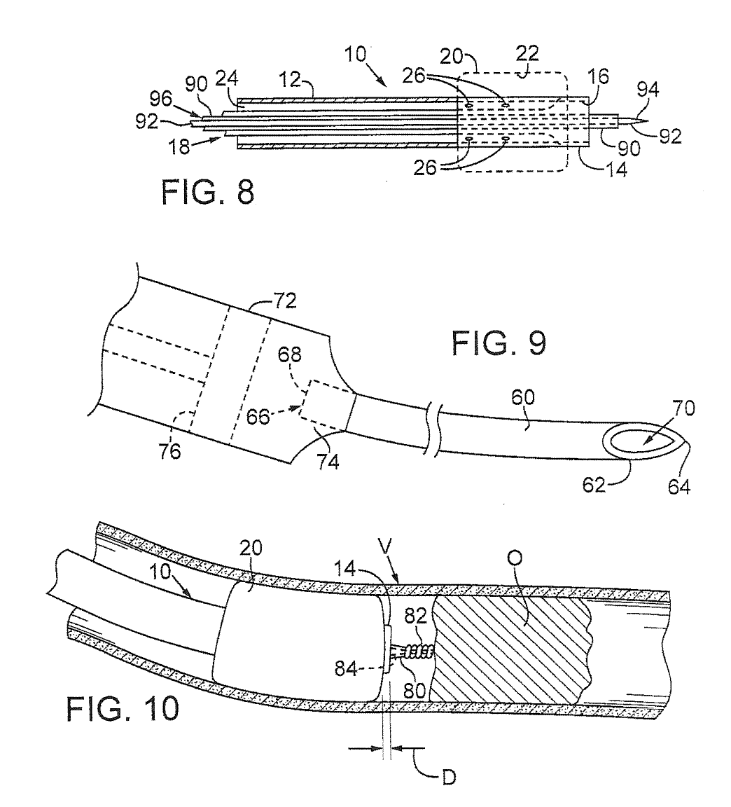

Another wire that may be used in catheter 10 is shown in FIG. 8, where a hollow wire 90 is inserted through lumen 18 of catheter 10. A needle wire 92 with a pointed tip 94 may be inserted through a lumen 96 of hollow wire 90. The relative positions of catheter 10, hollow wire 90, and needle wire 92 are under control of the physician, as for the wires described above. Thus, wires 90 and 92 may be used to infuse liquid and pierce through an occlusion as described above for wires 60 and 80.

Other wires may be used in conjunction with catheter 10 for the treatment of occlusions, for example, the Safe-Cross® RF Crossing Wire made by Intraluminal Therapeutics, Inc. of Carlsbad, Calif. Alternatively, a laser wire could be used.

Another wire that can be used in catheter 10 is made by LuMend, Inc. of Redwood City, Calif. Such a wire 100 is shown in FIGS. 13 and 14 with a blunt micro-dissection tool 102 at a distal end 104 of wire 100. Tool 102 includes two jaws 106, 108, which when closed, as seen in FIG. 13, form a generally blunt tip 110 that engages occlusion O. As shown in FIG. 14, jaws 106 and 108 may be opened to push the plaque and calcification apart, allowing tool 102 and wire 100 to be advanced through the occlusion.

Catheter 10 or other wires may be used prior to operation of the Intraluminal, laser, or LuMend wires to infuse liquid to dissolve the plaque and calcification as described above. Typically, when the wire tip has been passed all the way through occlusion O, a stent and/or balloon catheter may be advanced through occlusion O and expanded to reopen the blood vessel.

Catheter 10 is typically used with a balloon that inflates to a substantially cylindrical shape, as shown, e.g., in FIG. 3. Alternatively, the balloon may be provided with another shape suitable for the desired application. For example, as shown in FIG. 12, catheter 10 may include a balloon 20a that inflates to a substantially oval shape. These and other balloons typically are disposed on catheter 10 as for balloon 20 and may be used in providing treatment as described above. FIG. 12 shows hollow, needle-tipped wire 60 inserted through catheter 10, with distal tip 62 extending beyond catheter distal end 14, but other wires may be used with balloon 20a.

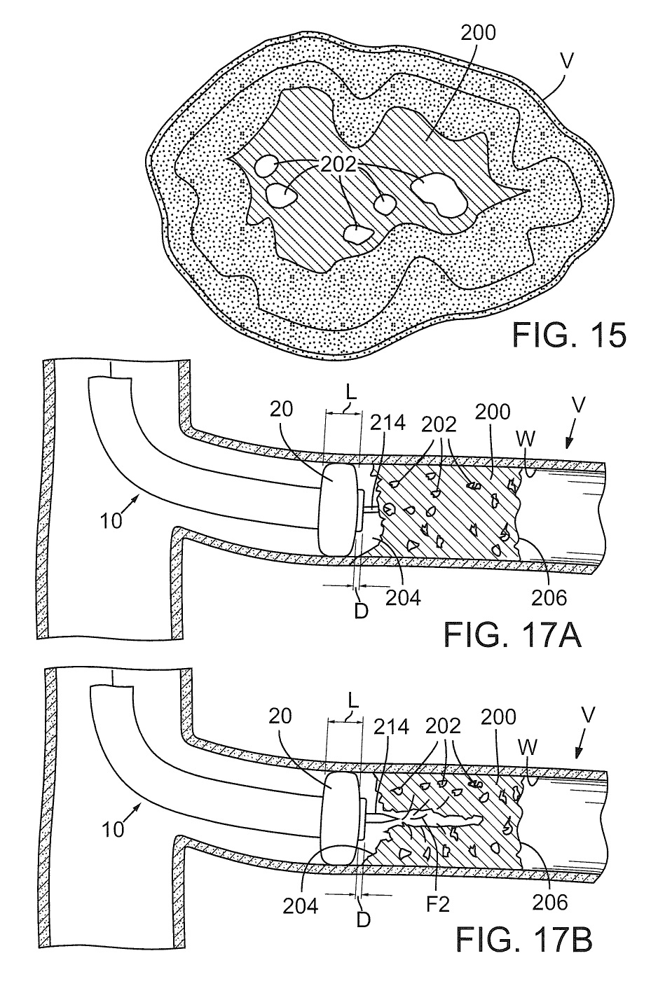

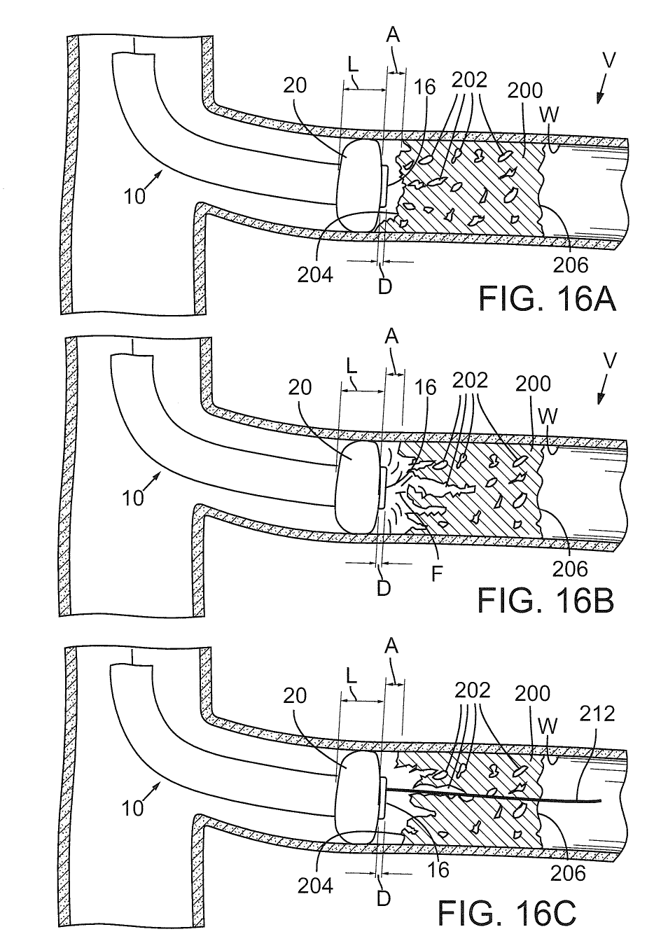

Another aspect involves exploiting existing characteristics of a total chronic occlusion (“CTO”) in order to obtain access to an area of a blood vessel distal the CTO. FIG. 15 depicts a cross-sectional view of a CTO 200 in a blood vessel V. CTO 200 includes one or more microchannels 202 that may be expanded to gain access through CTO 200. For example, microchannel 202 may be expanded to form a channel through CTO 200 into which a needle or other instrument can be inserted. Methods for performing such a procedure utilizing various occlusion-penetrating devices are depicted in FIGS. 16-18. For reference, CTO 200 has a proximal end 204 and a distal end 206 (see FIGS. 16-18).

When performing the methods depicted in FIGS. 16-18, it is advantageous to use a balloon 20 that has a length L that is less than existing balloons. For example, balloon 20 may have a length L that is no more than about 3 mm, and preferably no more than about 2 mm in length. Balloon 20 may be expandable to a diameter suitable to block a vessel V, such as 4 mm. It also may be advantageous to use a balloon that is elastomeric (also referred to as an elastomeric membrane), which is better suited for stabilizing instruments within a blood vessel than non-elastomeric balloons.

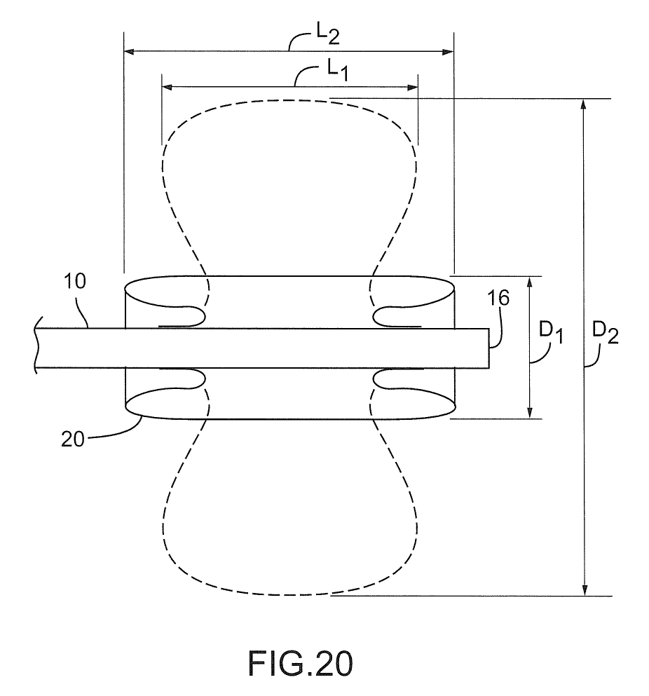

FIG. 20 depicts an example embodiment of a balloon 20 that may be used to stabilize a catheter within a blood vessel. In this example, the balloon 20 is mounted to catheter 10 so that when inflated, it folds underneath itself (as shown in phantom). As balloon 20 is inflated, its diameter increases from D₁ to D₂, which causes its length to decrease from L₂ to L₁. In one embodiment, balloon 20 has a length (L₂) of 2.5 mm when deflated and a length (L₁) of 2 mm when inflated. In some embodiments such as the one shown in FIG. 20, balloon is connected to an outer surface of catheter 10 along a portion of the outer surface that is shorter than the length of balloon 20 when deflated (L₂).

Referring now to FIG. 16A, a catheter 10 similar to those described above is inserted into a blood vessel V that is blocked by CTO 200, and first balloon 20 is inflated so that it substantially closes off the blood vessel V. When expanded, balloon 20 also serves to stabilize catheter 10 within blood vessel V so that catheter 10 is not forced backwards when force is applied to CTO 200. Although catheter 10 is shown in FIGS. 16A-C as being inserted so that balloon 20 is immediately adjacent CTO 200, it should be understood that balloon 20 may be positioned elsewhere in the blood vessel V, such as at the vessel's origin.

As seen in FIG. 16B, the occlusion-penetrating device is an indeflator (not shown), and the indeflator is operated by a physician to expel pressurized fluid F out of distal aperture 16 of catheter 10. The pressurized fluid F can be any number of solutions, such as contrast mixed with saline or a tissue-destroying substance such as collagenase or thrombolytic substances.

Where non-dangerous chemicals such as saline are used as the fluid F, balloon 20 may be inflated anywhere in the blood vessel V, including at the origin. However, where dangerous chemicals are used as the fluid F, it is preferable to insert catheter 10 to a position in very close proximity to CTO 200 so that when balloon 20 is inflated, the area A of the wall W of vessel V that is exposed to the chemical is minimized, thus reducing trauma to the vessel V.

As the fluid F is expelled from aperture 16 at high pressure, the fluid pressure within the blood vessel V increases. Microchannels 202 in CTO 200 offer the path of least resistance for the pressurized fluid F, and so the fluid F tends to fill the microchannels 202, causing them to expand as shown in FIG. 16B. The pressurized fluid F may expand a microchannel 202 so that it forms a channel at least partially (FIG. 16B), and sometimes entirely, through CTO 200. Then, as shown in FIG. 16C, a wire 212 is inserted through an expanded microchannel 202, allowing additional treatment devices (e.g., angioplasty balloons, stents) to be maneuvered down wire 212 through CTO 200.

FIGS. 17A-B depict an alternative method of exploiting microchannels 202 to gain access through CTO 200. Catheter 10 is inserted to a position adjacent CTO 200 and balloon 20 is inflated to prevent proximal fluid flow and to stabilize catheter 10 within the vessel V (as shown in FIG. 16A). Instead of injecting high-pressure fluid F from distal aperture 16, a hollow needle 214 with an opening adjacent a sharp tip of the needle is inserted through distal aperture 16 and into a position where the needle's sharp tip is distal of the proximal end 204 of CTO 200. As seen in FIG. 17A, needle 214 is inserted to a position where it can inject fluid (e.g., collagenase, thrombolytic substance) into microchannel 202. A physician then injects fluid F2, causing microchannel to expand as seen in FIG. 17B. Once microchannel 202 is expanded, a wire 212 or other similar device may be advanced into expanded microchannel 202 and all the way through occlusion 200 (as shown in FIG. 16C).

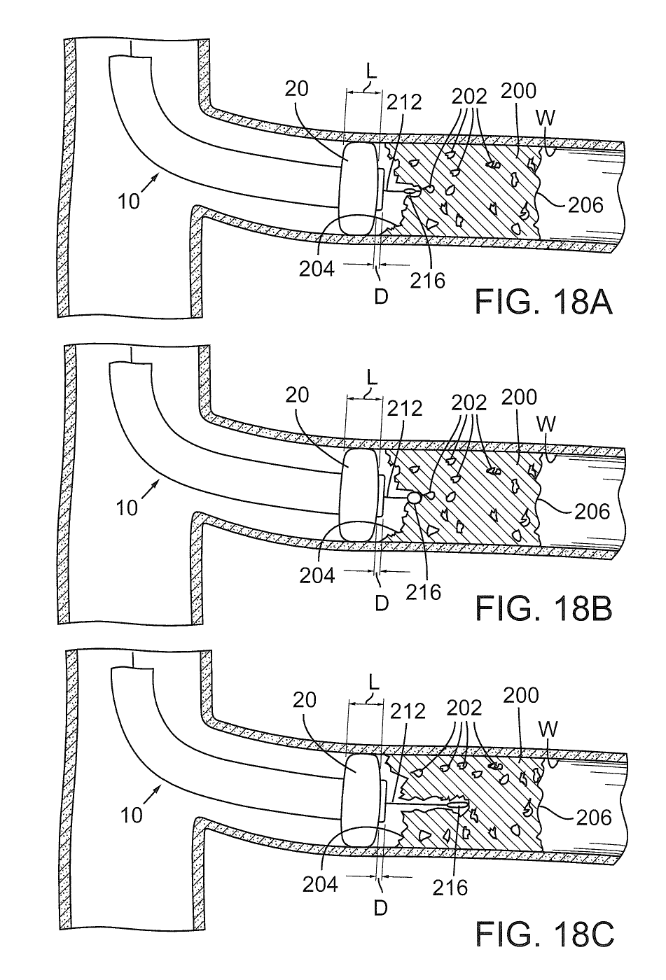

FIGS. 18A-C depict another method of exploiting microchannel 202 to gain access through CTO 200. Once again, catheter 10 is inserted and balloon 20 is expanded, as shown in FIG. 16A. However, instead of injecting fluid, a wire 212 having a very small balloon 216 disposed thereon is inserted out of distal aperture 16 and to a position where very small balloon 216 is within microchannel 202, as shown in FIG. 18A. Very small balloon 216 may have a length of about 0.5 mm to about 1 mm, and may be expanded to a diameter of between about 0.5 mm and 1 mm.

As shown in FIG. 18B, very small balloon 216 is expanded, which correspondingly expands microchannel 202. Very small balloon 216 is then deflated and wire 212 is advanced through the expanded microchannel 202, as shown in FIG. 18C, so that either very small balloon 216 may be expanded within another microchannel 202, or wire 212 may be simply advanced all the way through CTO 200 (see FIG. 16C).

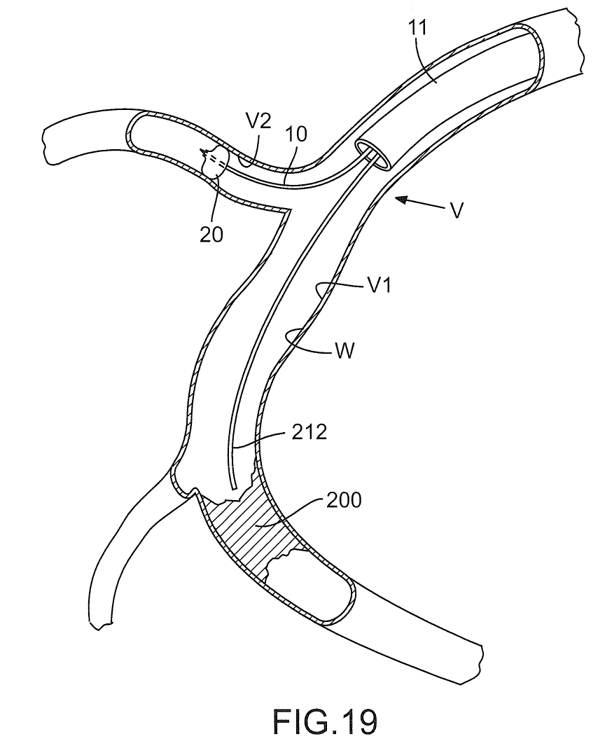

FIG. 19 depicts another method of stabilizing a support catheter 11 within a vessel system using an elastomeric balloon. CTO 200 is located in a main branch V1. Instead of being inserted into main branch V1, catheter 10 and balloon 20 are inserted from support catheter 11 into a side branch V2, where balloon 20 is inflated. An occlusion-penetrating device such as wire 212 is then advanced down the main branch V1 to treat CTO 200. Positioning catheter 10 and balloon within the side branch V2 anchors the entire treatment apparatus, including support catheter 11, within the vessel system. This allows a physician to apply considerable force to CTO 200 without fear of any of the devices being forced in a proximal direction. Alternatively, any of the methods depicted in FIGS. 16-18 may be used to exploit microchannels in CTO 200.

While various embodiments have been disclosed in their preferred forms, the specific embodiments thereof as disclosed and illustrated herein are not to be considered in a limiting sense as numerous variations are possible. Applicant regards the subject matter to include all novel and non-obvious combinations and subcombinations of the various elements, features, functions and/or properties disclosed herein. No single feature, function, element or property of the disclosed embodiments is essential. The following claims define certain combinations and subcombinations which are regarded as novel and non-obvious. Other combinations and subcombinations of features, functions, elements and/or properties may be claimed through amendment of the present claims or presentation of new claims in this or a related application. Such claims are also regarded as included within the subject matter of the present disclosure irrespective of whether they are broader, narrower, or equal in scope to the original claims.

I claim:

1. A method of treating an occlusion within a blood vessel, the method comprising:
inserting a catheter defining proximal and distal ends and a central lumen terminating at an aperture at the distal end into the blood vessel;
inflating an elastomeric membrane mounted on the catheter so that the elastomeric membrane substantially closes off the blood vessel and stabilizes the position of the catheter within the blood vessel, the elastomeric membrane including a proximal end and an opposite distal end;
operating an occlusion-penetrating device to expand an existing microchannel in the occlusion to form a channel at least partially through the occlusion.

2. The method of claim 1, wherein the elastomeric membrane is disposed immediately adjacent to the aperture at the distal end of the catheter and is inflated to no more than 3 to 5 mm in length.

3. The method of claim 2, wherein the elastomeric membrane is inflated to no more than about 2 to 5 mm in length.

4. The method of claim 2, wherein the elastomeric membrane is inflated so that the distal end of the elastomeric membrane is no more than about 1 mm from the distal end of the catheter.

5. The method of claim 2, wherein the elastomeric membrane is inflated so that the distal end of the elastomeric membrane is no more than about 0.5 mm from the distal end of the catheter.

6. The method of claim 1, wherein the occlusion-penetrating device is a fluid injection device, and the method further comprises:
inserting the catheter so that the elastomeric membrane is positioned at an origin of the blood vessel; and
operating the fluid injection device to expel a treatment fluid out of the aperture at the distal end of the catheter at a pressure sufficient to cause an existing microchannel in the occlusion to expand to form a channel at least part of the way through the occlusion.

7. The method of claim 1, wherein the occlusion-penetrating device is a RF wire insertable through the central lumen of the catheter, and wherein the method further comprises operating the RF wire to apply RF energy to the occlusion.

8. The method of claim 1, wherein the occlusion-penetrating device is a hollow needle with an opening adjacent a sharp tip of the needle, and wherein the method further comprises inserting the sharp tip into a microchannel of the occlusion and injecting solution to cause the microchannel to expand to form a channel at least part of the way through the occlusion.

9. The method of claim 1, wherein the occlusion-penetrating device is a wire with a second membrane that is no larger than 0.5 mm in length, and wherein the method further comprises:
inserting the wire to a position where the second membrane is within a microchannel of the occlusion; and
inflating the second membrane to expand the microchannel to form a channel at least part of the way through the occlusion.

10. The method of claim 1, further comprising inserting a wire into the channel formed from the expanded microchannel to penetrate the entire occlusion.

11. A method of treating an occlusion within a blood vessel, the method comprising:
inserting a first catheter with a first lumen terminating at a distal aperture into the blood vessel;
inserting a second catheter with a second lumen through the first lumen of the first catheter and out of the distal aperture; and
inflating an elastomeric membrane mounted on the second catheter immediately adjacent the distal end so that the elastomeric membrane substantially closes off the blood vessel and maintains a position of the first catheter in the blood vessel;
operating an occlusion-penetrating device to penetrate the occlusion.

12. The method of claim 1, wherein inserting the second catheter includes inserting the second catheter into a side branch of the blood vessel; and inflating the elastomeric membrane includes inflating the membrane so it substantially closes off the side branch of the blood vessel.

13. The method of claim 11, wherein the elastomeric membrane is inflated to no more than about 2 mm in length.

14. The method of claim 11, wherein the occlusion-penetrating device is a hollow needle with an opening adjacent the needle's sharp tip, and wherein the method further comprises:
inserting the sharp tip into a microchannel of the occlusion; and
injecting solution to cause the microchannel to expand to form a channel at least part of the way through the occlusion.

15. The method of claim 11, wherein the occlusion-penetrating device is a wire with a second membrane that is no larger than 0.5 mm in length, and wherein the method further comprises:
inserting the wire to a position where the second membrane is within a microchannel of the occlusion; and
inflating the second membrane to expand the microchannel to form a channel at least part of the way through the occlusion.

16. The method of claim 14, further comprising inserting a wire through the channel formed from the expanded microchannel to penetrate the entire occlusion.

17. The method of claim 15, further comprising inserting a wire through the channel formed from the expanded microchannel to penetrate the entire occlusion.

18. A catheterization system for treatment of an occlusion within a blood vessel, the system comprising:
a catheter defining a proximal end, a distal end, a central lumen interconnecting the ends, and an aperture at the distal end, the catheter insertable into the blood vessel to a position proximal to the occlusion;
an elastomeric membrane mounted on the catheter and disposed immediately adjacent to the aperture at the distal end of the catheter, the elastomeric membrane including a proximal end and an opposite distal end and being inflatable to substantially close off the blood vessel and to stabilize the catheter in the blood vessel; and
an occlusion-penetrating device operable to form a channel through the occlusion.

19. The catheter system of claim 18, wherein the elastomeric membrane has a length that decreases as the elastomeric membrane is inflated.

20. The catheter system of claim 19, wherein the elastomeric membrane is connected to an outer surface of the catheter along a portion of the outer surface that is shorter than the length of the elastomeric membrane when deflated.
